(19) United States
(12) Patent Application Publication
(10) Pub. No.: US 2017/0332909 A1
(43) Pub. Date: Nov. 23, 2017

(54) OBJECT INFORMATION ACQUIRING APPARATUS

(71) Applicant: CANON KABUSHIKI KAISHA, Tokyo (JP)

(72) Inventors: Kenichi Nagae, Yokohama-shi (JP); Robert A Kruger, Oriental, NC (US)

(73) Assignee: CANON KABUSHIKI KAISHA, Tokyo (JP)

(21) Appl. No.: 15/522,905

(22) PCT Filed: Nov. 2, 2015
(86) PCT No.: PCT/JP2015/081420

§ 371 Date: Apr. 28, 2017

Related U.S. Application Data
(60) Provisional application No. 62/077,369, filed on Nov. 10, 2014.

(51) Int. Cl. A61B 5/00; A61B 8/08; A61B 8/00

(52) U.S. Cl. CPC A61B 5/0035; A61B 8/4494; A61B 5/0095; A61B 8/0825; A61B 8/0858

(57) ABSTRACT

Provided is an object information acquiring apparatus, having: an ultrasound transmitting element; a plurality of transducers each detecting a first acoustic wave generated by light, which is radiated into an object, and outputting a first electric signal, and detecting a second acoustic wave generated by an ultrasound wave, which is transmitted from the ultrasound transmitting element and which is scattered inside the object, and outputting a second electric signal; a support supporting the plurality of transducers so that directivity axes of the transducers are concentrated; and a processor acquiring property information on the object based on the first electric signal and the second electric signal respectively.

### TECHNICAL FIELD

The present invention relates to an object information acquiring apparatus.

### BACKGROUND ART

Research on an optical imaging apparatus that irradiates an object (e.g. living body) with light from a light source (e.g. laser) and images information inside the object acquired based on the entered light is actively ongoing in medical fields. Photoacoustic imaging (PAI) is one optical imaging technique. In photoacoustic imaging, pulsed light generated from a light source radiated into an object, and an acoustic wave (typically an ultrasound wave) generated from an object tissue, which absorbed the energy of the pulsed light propagated and diffused inside the object, is detected. Then, based on the detected signal, internal information of the object is imaged.

Recently using this photoacoustic imaging, pre-clinical research to image the vascular image of small animals and clinical research to apply this principle to diagnose breast cancer or the like are actively ongoing.

Patent Literature 1 discloses an apparatus that generates ultrasound signals by radiation of an electromagnetic wave from an electromagnetic radiation source, and includes a receiving element group to receive the ultrasound signals. This receiving element group is disposed on a spherical surface, whereby better imaging can be performed regardless the direction of the absorber inside the object.

Further, Patent Literature 2 discloses an apparatus that acquires a photoacoustic image and an ultrasound image.

### CITATION LIST

### Patent Literature

PTL1: U.S. Pat. No. 5,713,356

PTL2: Japanese Patent No. 4406226

### SUMMARY OF INVENTION

### Technical Problem

However, thus far an apparatus that can perform photoacoustic imaging regardless the direction of the absorber inside the object, and that can also acquire an ultrasound image, has not been disclosed.

With the foregoing in view, it is an object of the present invention to provide an apparatus that can perform photoacoustic imaging regardless the direction of the absorber inside the object, and can also acquire an ultrasound image.

### Solution to Problem

The present invention provides an object information acquiring apparatus comprising:

a light source;

an ultrasound transmitting element;

a plurality of transducers each configured to detect a first acoustic wave generated by light, which is from the light source and which is radiated on an object, and output a first electric signal, and detect a second acoustic wave generated by an ultrasound wave, which is transmitted from the ultrasound transmitting element and which is scattered inside the object, and output a second electric signal;

a supporter configured to support the plurality of transducers so that directivity axes of the transducers are concentrated; and

a processor configured to acquire property information on the object based on the first electric signal and the second electric signal respectively.

### Advantageous Effects of Invention

According to the present invention, an apparatus that can perform photoacoustic imaging regardless the direction of the absorber inside the object, and can also acquire an ultrasound image, can be provided.

Further features of the present invention will become apparent from the following description of exemplary embodiments (with reference to the attached drawings).

### BRIEF DESCRIPTION OF THE DRAWINGS

FIG. 1 schematically shows an example of a configuration of a photoacoustic apparatus according to the present invention.

FIG. 2 schematically shows an ultrasound image reconstruction according to the present invention.

FIG. 3 shows the transmission timings of pulsed light and an ultrasound wave according to the present invention.

FIG. 4 shows another example of a movement locus of a supporter according to the present invention.

FIG. 5 schematically shows another embodiment of the present invention.

FIG. 6A and FIG. 6B show examples of the movement locus according to the present invention.

### DESCRIPTION OF EMBODIMENTS

Preferred embodiments of the present invention will now be described with reference to the drawings. Dimensions, materials, shapes of the components and relative positions and the like of the preferred embodiments described herein below can be appropriately changed depending on the configuration and various conditions of the apparatus to which the invention is applied, and are not intended to limit the scope of the invention to the following description.

The present invention relates to a technique to detect an acoustic wave propagated from an object, and to generate and acquire property information inside the object. Therefore the present invention is understood as an object information acquiring apparatus, a control method thereof, an object information acquiring method, and a signal processing method. The present invention can also be understood as a program that causes an information processor, which includes such hardware resource as a CPU, to execute these methods, and a storage medium storing this program. The present invention is also understood as an acoustic measurement apparatus and a control method thereof.

The present invention can be applied to an object information acquiring apparatus that uses a photoacoustic tomography technique, to irradiate an object with light (electromagnetic wave), and receives (detects) an acoustic wave that is generated inside the object or at a specific position on the surface of the object according to the photoacoustic effect, and is propagated. Such an apparatus, which acquires the property information inside the object in the form of, for example, image data or property distribution information based on the photoacoustic measurement, can also be called a “photoacoustic imaging apparatus” or simply a “photoacoustic apparatus”.

The property information in the photoacoustic apparatus is, for example, a generation source distribution of an acoustic wave generated by the light radiation, an initial sound pressure distribution inside the object, a light energy absorption density distribution or absorption coefficient distribution derived from the initial sound pressure distribution, or a concentration distribution of a substance constituting a tissue. The concentration of a substance is, for example, an oxygen saturation, an oxyhemoglobin concentration, a deoxyhemoglobin concentration, a total hemoglobin concentration or the like. Total hemoglobin concentration is a total of the oxyhemoglobin concentration and the deoxyhemoglobin concentration. The distributions of fat, collagen, water and the like can also be a subject of property information. The property information may be determined not as numerical data but as distribution information at each position inside the object. In other words, the object information may be distribution information, such as an absorption coefficient distribution and an oxygen saturation distribution.

The present invention can also be applied to an apparatus utilizing an ultrasound echo technique to transmit an ultrasound wave into an object, and receive a reflected wave (echo wave) reflected inside the object, whereby the object information is acquired as image data. In the case of an apparatus utilizing the ultrasound echo technique, the acquired object information is information reflecting the difference of acoustic impedance among tissues inside the object.

An object information acquiring apparatus according to a typical embodiment of the present invention can acquire both the property information originating from the photoacoustic wave of an object, and the property information originating from the ultrasound echo of the object.

An acoustic wave referred to in the present invention is typically an ultrasound wave, and includes an elastic wave which is called a “sound wave” or an “acoustic wave”. An acoustic wave generated by the photoacoustic effect is called a “photoacoustic wave” or a “light-induced ultrasound wave”. An electric signal (reception signal) converted from an acoustic wave by a probe is called an “acoustic signal”, and an acoustic signal originating from the photoacoustic wave in particular is called a “photoacoustic signal”.

An object used in the present invention can be a breast of a living body. The object, however, is not limited to this, but may be other segments of a living body or a non-biological material.

<General Configuration of Photoacoustic Apparatus>

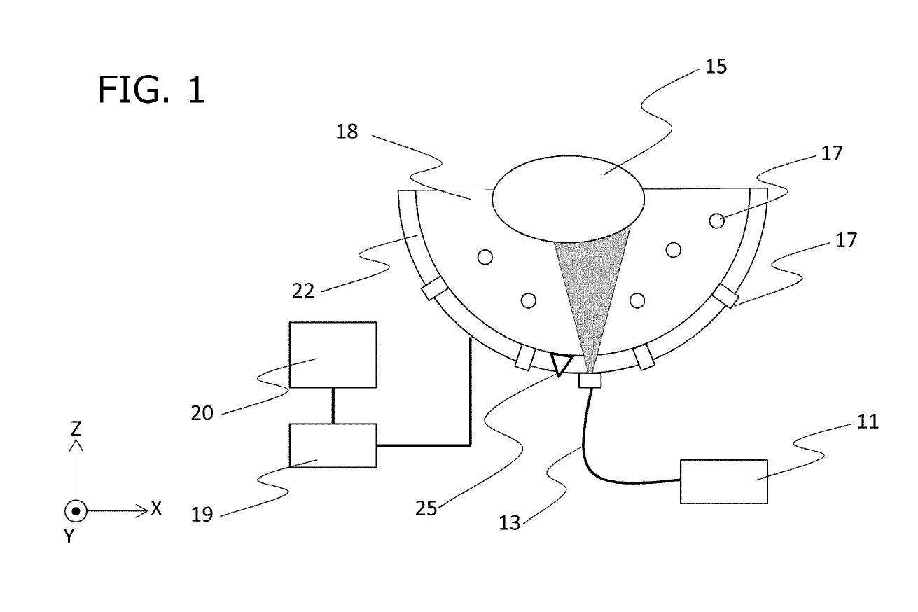

A configuration of the photoacoustic apparatus according to this embodiment will be described with reference to FIG. 1.

The photoacoustic apparatus of this embodiment has a light source 11, an optical transmission system 13, a plurality of transducers 17 supported by the supporter 22, a computer 19, a display apparatus 20, an ultrasound transmitting element 25, and an acoustic matching material 18 which exists between the object 15 and the plurality of transducers 17.

First the photoacoustic imaging executed in this apparatus will be described.

Pulsed light emitted from the light source 11 is processed into a desired light distribution shape by the optical transmission system 13 constituted by a lens, a mirror, an optical fiber, a diffusion plate and the like, and is guided and radiated into an object 15, such as a living body. At a timing when the pulsed light is radiated, the pulsed light almost simultaneously reaches the entire inside of the object 15. If a part of the energy of the pulsed light propagated inside the object 15 is absorbed by a light absorber (which becomes a sound source), such as blood vessels containing considerable hemoglobin, a photoacoustic wave (typically an ultrasound wave) is generated by the thermal expansion of the light absorber. The photoacoustic wave propagates inside the object 15 and acoustic matching material 18, and reaches a plurality of transducers 17 supported by the supporter 22. The plurality of transducers 17 receives this photoacoustic wave and converts it into a plurality of electric signals.

Then appropriate amplification processing and digital processing are performed on the plurality of electric signals outputted from the plurality of transducers 17, whereby a plurality of photoacoustic signals is outputted to the computer 19. The computer 19 is a processor that performs the reconstruction processing on the photoacoustic digital signals, and generates a photoacoustic image showing the inside of the object. For this reconstruction processing, a known reconstruction method, such as Universal Back Projection (UBP) and Filtered Back Projection (FBP), can be used.

The photoacoustic image generated by the computer 19 is outputted to the display apparatus 20, and the inputted photoacoustic image is displayed on the display apparatus 20.

Now the ultrasound imaging in this apparatus will be described.

An ultrasound wave transmitted from the ultrasound transmitting element 25 is reflected and scattered according to the acoustic impedance distribution inside the object. The scattered ultrasound wave propagates through the object 15 and acoustic matching material 18, and reaches the plurality of transducers 17 which is supported by the supporter 22. The plurality of transducers 17 receives this ultrasound wave and converts it into a plurality of electric signals.

Then appropriate amplification processing and digital processing are performed on the plurality of electric signals outputted from the plurality of transducers 17, whereby a plurality of ultrasound digital signals is outputted to the computer 19. The computer 19 performs the later mentioned ultrasound image reconstruction processing on the ultrasound digital signals, and generates the ultrasound image showing the inside of the object.

The ultrasound image generated by the computer 19 is outputted to the display apparatus 20, and the inputted ultrasound image is displayed on the display apparatus 20.

<Each Component of Photoacoustic Apparatus>

Now each component of the photoacoustic apparatus according to this embodiment will be described in detail.

(Light Source 11)

The light source 11 supplies light energy to the object so as to generate the photoacoustic wave. If the object is a living body, the light source 11 radiates light having a specific wavelength that is absorbed by a specific component out of the components constituting the object. It is preferable to use a wavelength-variable light source. For the light source, a pulsed light source that can generate pulsed light in a several nano to several hundred nano second order as the radiation light is preferred. In concrete terms, a light source which can generate light having a 10 to 100 nano second pulse width is preferred, in order to generate the photoacoustic wave efficiently. For the light source, laser is desirable because of its high output, but a light emitting diode or the like may be used instead of laser. For the laser, various lasers can be used, such as a solid-state laser, a gas laser, a fiber laser, a dye laser and a semiconductor laser. The timing of light radiation, waveform, intensity or the like can be controlled by a light source controller, which is not illustrated. If the object is a living body, the wavelength of the light source to be used is preferably a wavelength with which the light can be propagated into the internal area of the living body. In concrete terms, such a wavelength is 500 nm or more, 1200 nm or less.

The light source 11 may be provided separately from the photoacoustic apparatus. The light source 11 may be constituted by a single light source, or may be constituted by a plurality of light sources.

(Optical Transmission System 13)

The pulsed light radiated from the light source 11 is normally processed into a desired light distribution shape by optical components, such as a lens and mirror, and is guided into the object 15. The pulsed light may be propagated using an optical wave guide, which is an optical fiber, a bundled optical fiber or an articulating arm constituted by a lens barrel in which a mirror and other components are integrated, and these members are also regarded as the optical transmission system 13. The optical transmission system 13 may also include, for example, a mirror to reflect light, a lens to collect or expand light or to change the shape of the light, and a diffusion plate to diffuse light. Any such optical component may be used if the pulsed light emitted from the light source can be processed into a desired shape and be radiated into the object 15 in this state. It is preferable that the light is expanded to a certain area, instead of being collected by a lens, since the diagnostic region in the object can be expanded.

(Object 15 and Light Absorber)

The object 15 and the light absorber will be described here, although neither are part of the photoacoustic apparatus. The photoacoustic apparatus according to this embodiment is primarily used for the diagnosis of malignant tumors and vascular diseases of humans and animals, follow up observation of chemotherapy or the like. Therefore a possible object 15 would be a target segment for diagnosis, such as a breast, a finger, a limb or the like of a human or animal. A light absorber inside the object is a substance which has a relatively high absorption coefficient within in the object 15, and if the measurement target is a human body, the light absorber corresponds to oxyhemoglobin, deoxyhemoglobin, blood vessels containing a high amount of these hemoglobins, a neo-vascularity or the like. A light absorber on the surface of the object 15 is melanin or the like. However, other substances, including fat, water and collagen, can be a light absorber in the human body if an appropriate wavelength of the light is selected.

(Transducer 17)

The transducer 17 receives an acoustic wave (photoacoustic wave or scattered ultrasound wave) generated in the object, and converts the acoustic wave into an electric signal, which is an analog signal. Any transducer, such as a transducer using the piezoelectric phenomenon, a transducer using the resonance of light, or a transducer using the change of capacitance, can be used for the transducer 17, only if the acoustic wave can be detected. In this embodiment, a plurality of transducers 17 is disposed. By using such multi-dimensionally arrayed elements, an acoustic wave can be simultaneously received at a plurality of locations, whereby measurement time can be decreased, and the influence of the vibration of the object 15 or the like can be reduced.

This embodiment is described using an example of receiving a photoacoustic wave and a scattered ultrasound wave using a same transducer 17. However, these acoustic waves may be received using different transducers respectively. In this case, if a transducer having a frequency characteristic suitable for each acoustic wave is used respectively, the SN ratio improves, and therefore image quality improves. The size of the transducer may be changed according to the spatial resolution required for the photoacoustic image and the ultrasound image respectively.

(Supporter 22)

The supporter 22 is a member that supports a plurality of transducers 17 along the supporter 22. FIG. 1 is a cross-sectional view of the supporter 22 when the supporter 22 is sectioned by the x-z plane. FIG. 1 shows both the transducers 17 located on the cross-section of the support, and the transducers 17, of which tips are seen through the inner wall of the support.

It is preferable that the supporter 22 supports the plurality of transducers 17 such that the transducers 17 are arranged on a closed surface surrounding the object 15. However, if the object is a human body, for example, it is difficult to arrange a plurality of transducers 17 on the entire closed surface surrounding the object. In such a case, it is preferable to arrange a plurality of transducers 17 on the hemispherical surface of the supporter 22, which has an opening, as shown in this embodiment.

It is preferable that the plurality of transducers 17 on the supporter 22 is arranged such that sampling at equal intervals is possible in the k-space. For example, it is preferable that the plurality of transducers 17 is arranged spirally, as disclosed in Patent Literature 1.

Generally the reception sensitivity of a transducer is highest in the normal line direction of the reception plane (surface). By concentrating the axis along the direction in which the reception sensitivity is highest (hereafter called “directivity axis”) of the plurality of transducers 17 toward a curvature center point of the hemispherical shape, a region which can be visualized at high precision is formed in an area around the curvature center point. Particularly in this embodiment, the plurality of transducers 17 is disposed such that each directivity axis crosses at the curvature center of the hemisphere. Then the resolution of the region where the directivity axes are concentrated can be enhanced. In this description, this region of which resolution is enhanced is called “high resolution region 23”. In this embodiment, the high resolution region 23 is a region from the highest resolution point to the line where the resolution is half the highest resolution. The directivity axis of each transducer need not always cross with each other, as long as the directivity axes can be concentrated into a specific region and a desired high resolution region 23 can be formed.

FIG. 1 is an example of the arrangement of the transducers, but the arrangement is not limited to this. All that is required for the arrangement of transducers is that the directivity axes are concentrated to a desired region where a desired high resolution region can be formed. In other words, it is sufficient that the plurality of transducers 17 is arranged along the curved surface, such that a desired high resolution region is formed. Further, “the curved surface” in this description includes a true sphere and a spherical surface having an opening, such as a hemisphere. A surface which has unevenness thereon but which can roughly be regarded as a spherical surface, or a surface on an ellipsoidal (a form generated by expanding an ellipse three-dimensionally, and of which surface is a quadratic surface) which can roughly be regarded as a sphere, are also included in the “curved surface”.

When the plurality of transducers 17 is arranged along the supporter 22, having an arbitrary cross-section of a sphere, the directivity axes are concentrated to the center of the curvature of the shape of the support. The hemispherical supporter 22 described in this embodiment is also an example of the support having an arbitrary cross-section of a sphere. In this description, a shape having an arbitrary cross-section of a sphere is called a “shape based on a sphere”. The plurality of transducers supported by the support which has a shape based on a sphere is supported on the spherical surface.

Further, for example, other curved or piecewise linear surfaces could also be used as the supporter 22.

It is preferable that the supporter 22 has a space in which the acoustic matching material 18 is filled.

By installing the transducers 17 in an arrangement to surround the object 15 like this, a photoacoustic wave generated inside the object can be received from various directions. Therefore the photoacoustic image can be reconstructed in a state of reducing influences depending on the direction of the absorber inside the object, and a photoacoustic image, in which the visibility of an absorber (e.g. blood vessels) in the extending direction is improved, can be provided.

(Acoustic Matching Material 18)

The acoustic matching material 18 is an impedance matching material which fills the space between the object 15 and the plurality of transducers 17, and acoustically couples the object 15 and the plurality of transducers 17. A preferable material of the acoustic matching material 18 is a material which has acoustic impedance that is close to those of the object 15 and the transducers 17, and which transmits the pulsed light. For example, water, caster oil, gel or the like is used for the acoustic matching material 18.

(Computer 19)

The computer 19 performs predetermined processing on each electric signal outputted from the plurality of transducers 17. The computer 19 also controls the operation of each component of the photoacoustic apparatus.

(Display Apparatus 20)

The display apparatus 20 is an apparatus to display image data outputted by the computer 19. For the display unit, a liquid crystal display is typically used, but another type of display, such as a plasma display, an organic EL display and an FED, may be used instead. The display apparatus 20 may be provided separately from the photoacoustic apparatus.

(Ultrasound Transmitting Element 25)

The ultrasound transmitting element 25 is an element to transmit an acoustic wave to the object according to the inputted electric signal. Any element that can transmit an acoustic wave, such as an element using the piezoelectric phenomenon or an element using a change in capacitance, may be used for the ultrasound transmitting element 25. In this embodiment, only one ultrasound transmitting element 25 is used, but a plurality of ultrasound transmitting elements 25 may be disposed in the supporter 22, and in this case, the plurality of ultrasound transmitting elements 25 may be sequentially switched for transmitting the acoustic wave or may be driven simultaneously. If the ultrasound transmitting elements 25 are sequentially switched, scattered signals of ultrasound waves transmitted in various directions can be received, hence such effects as an improvement of performance in drawing the contour of a structure inside the object 15 and a reduction in speckles can be acquired. If the ultrasound transmitting elements 25 are simultaneously driven, an improvement in image quality is expected because of an improvement in the SN ratio due to an improvement in the transmission energy.

In this embodiment, an example of disposing the ultrasound transmitting element 25 separately from the plurality of transducers 17 used for reception is described, but the plurality of transducers 17 used for reception may also serve the function of the ultrasound transmitting elements 25. If the plurality of transducers 17 serve the function of the ultrasound transmitting elements 25, more transducers 17 can be disposed on the supporter 22, and such an effect as an improvement in the SN ratio and reduction of artifacts can be acquired because of the increase in the number of elements.

<Reconstruction Processing>

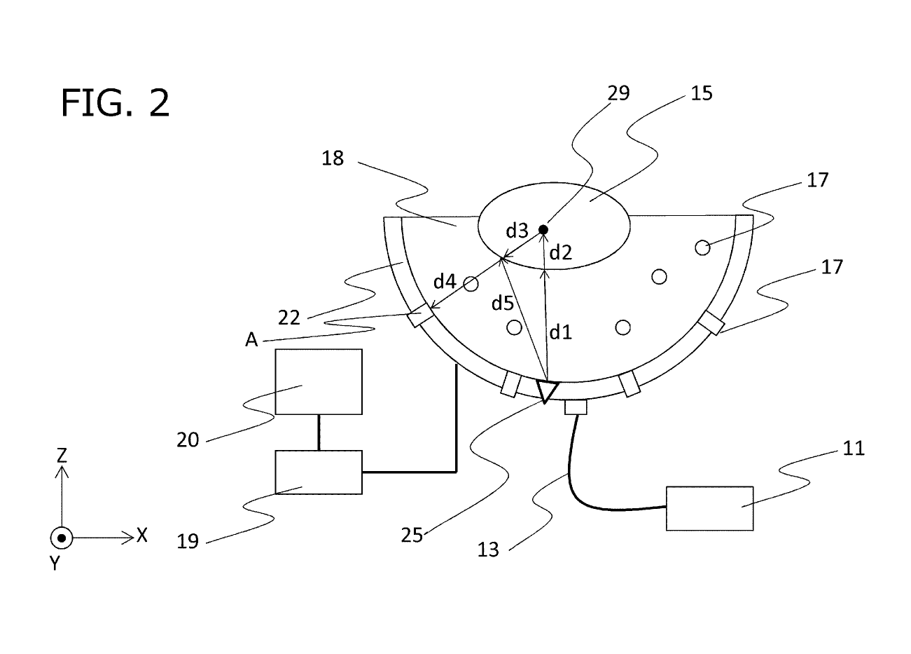

Now the ultrasound image reconstruction processing for the ultrasound digital signals executed by the computer 19 will be described with reference to FIG. 2.

Three-dimensional (3D) images of ultrasound are formed based on a filtered back projection approach, also referred to as “delay and sum”. The approach requires knowledge of the pulse-echo delay times, t(r), between when a transmit pulse is initiated in response to a “trigger” signal, and when it is detected by each transducer, after having been backscattered from each location (r) 29 within the tissue, as illustrated in FIG. 2. The geometry for this pulse-echo interaction is illustrated in FIG. 2. In this example, the bowl-array is filled with water with a known velocity of sound (V(water)). In general, the velocity of sound in the tissue (V(tissue)) differs from that for water. The relationship between the pulse-echo delay times and the imaging geometry is given as:

$$t ( r ) = \frac{d 1 + d 4}{V ( water )} + \frac{d 2 + d 3}{V ( tissue )} \qquad [ Math . 1 ]$$

where d1+d2 is the distance between the transmit transducer and a location within the tissue, and d3+d4 is the distance between each receive transducer and the same breast tissue location.

For understanding the image reconstruction process, the following definitions are introduced: hi(t): pulse-echo response of each transmit-receive transducer pair. This function can be measured by transmitting an acoustic wave off of a flat, metal plate and recording the resulting echo for the i-th receive transducer.

si(t): temporal signal recorded at transducer i following a transmit pulse.
Hi(w): FFT(hi(t)) is the Fourier Transform of the pulse-echo responses, where w is acoustic angular frequency.
Si(w): FFT(si(t)) is the Fourier Transform of the recorded temporal signals.

### Fili(w):

$${( \vert \frac{w}{{w}_{c}} \vert )}^{α} \frac{Apodize ( w , {w}_{c} )}{Hi ( w )} \qquad [ Math . 2 ]$$

is a filter function, where w<wC, 1≦α≦2, and wc is an upper bandwidth limit to the acoustic angular frequency.

The apodizing function is well known to those skilled in the art of computed tomography and is used to roll off the filter response smoothly at wc. An example function is

$$\frac{1 + cos ( π \frac{w}{{w}_{c}} )}{2} \qquad [ Math . 3 ]$$

s*i (t)=IFFT[Fili(w)Si (w)] is the filtered temporal signal recorded at transducer i following a transmit pulse, where IFFT is the inverse Fourier Transform.
The 3D integrated backscatter image is then computed as:

|(r)|Σᵢs*i(t(r))|*P(r), [Math. 4]

Where “∥” denotes absolute value, P(r) is a 3D smoothing filter, e.g., a 3D Gaussian, and “*” denotes 3D convolution.

By performing ultrasound image reconstruction like this, three-dimensional information of the inside of the object can be acquired, just like the case of a photoacoustic image.

In the case of reconstructing a photoacoustic image, the time required for the pulsed light to diffuse inside the object is assumed to be very short compared with the time required for a photoacoustic wave to reach from inside the object to the plurality of transducers 17, as mentioned above. Therefore in the reconstruction, it is sufficient if the distance d3+d4 in FIG. 2 is considered, and the arrival time, from when the pulsed light is radiated to the object, can be calculated by d3/V(tissue)+d4/V(water).

As mentioned, by using the same velocity of sound in the ultrasound image reconstruction and photoacoustic image reconstruction, the positions of the two types of images can be aligned accurately.

<Pulsed Light and Ultrasound Wave Timings>

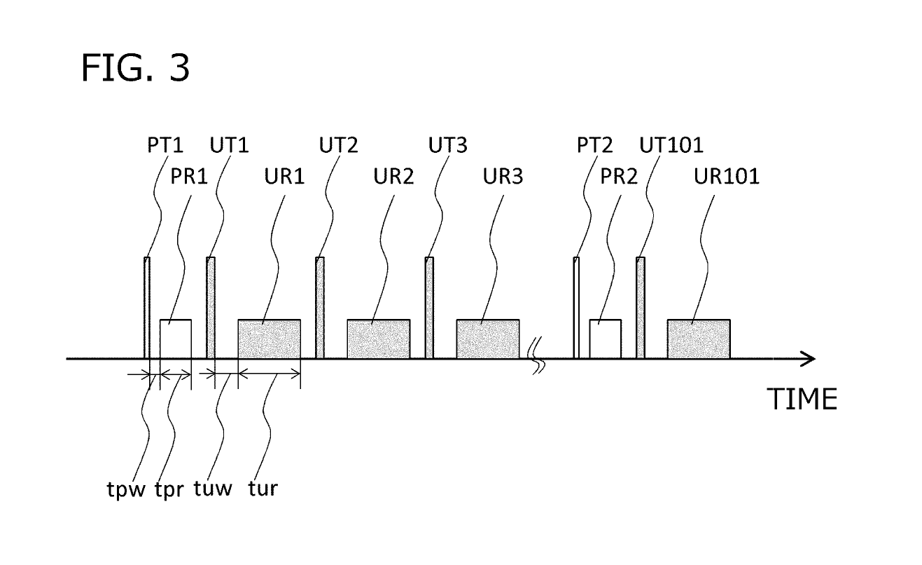

Now the transmitting timings and receiving timings of the pulsed light and the ultrasound wave will be described with reference to FIG. 3.

In FIG. 3, PT1 and PT2 denote the timings when the object 15 is radiated with the pulsed light, and PR1 and PR2 denote the periods when the photoacoustic wave, generated from the object, is received. UT1, UT2 and UT3 denote the timings to transmit the ultrasound wave to the object 15, and UR1, UR2 and UR3 denote the periods when the ultrasound wave, which scattered from the object 15, is received.

When a high output light source is used for the photoacoustic wave apparatus, the repeat frequency of the pulsed light is typically 10 Hz to 40 Hz. Therefore the radiation interval of the two beams of pulsed light is 25 msec to 100 msec. For example, if the radius of the supporter 22 is 150 mm, and the velocity of sound is 1500 m/sec for both a living body and water, then the time required from the radiation of the pulsed light into the object 15 to the completion of reception is at most 200 microseconds, which is sufficiently short compared with the radiation interval of the pulsed light. Therefore using the time of radiation intervals of the pulsed light, the ultrasound wave is transmitted/received during this period. The time required from the transmission of the ultrasound wave to the completion of reception is 400 microseconds, which is about double that of the case of the photoacoustic wave, since time for the ultrasound wave to return to inside the object is required, unlike the case of the photoacoustic wave.

In the present invention, the acoustic matching material 18 is filled inside the supporter 22, hence the reception start timing is different for each acoustic wave. For example, if one example is shown in use of FIG. 2, the light-induced ultrasound wave from the object 15 reaches the transducer A after the pulsed light is radiated becomes d4/V(water). On the other hand, the scattered ultrasound wave from the object 15 reaches the transducer A after the ultrasound wave is transmitted becomes (d4+d5)/V(water).

As described above, in the situation of acquiring the photoacoustic image and acquiring the ultrasound image, a difference is generated between the reception timings of each signal. Therefore processing to save memory and reduce the computer size, and to efficiently utilize processing capacity can be performed. In concrete terms, the period tpw from the radiation of the pulsed light to the start of receiving the photoacoustic digital signal used for reconstruction or to the start of recording [the photoacoustic digital signal] to memory, is differentiated from the period tuw from the transmission of the ultrasound wave to the start of receiving the ultrasound digital signal or the start of receiving the ultrasound digital signal to memory. Typically tpw>tuw is satisfied. Further, the period tpr of receiving the photoacoustic digital signal used for reconstructing the photoacoustic image or recording the photoacoustic digital signal to memory is differentiated from the period tur of receiving the ultrasound digital signal used for reconstructing the ultrasound image or recording the ultrasound digital signal to memory. Typically tpr<tur is satisfied.

By performing this control, an apparatus of which processing load is reduced can be provided.

The elements to transmit the ultrasound wave may be switched at UT1, UT2, UT3 or the like. If such ultrasound transmission is performed, a scattered ultrasound wave when the ultrasound wave is transmitted from various directions can be received, and an image, in which performance of drawing the contour of the structure inside the object is improved and interference of speckles is reduced, can be provided.

### Another Embodiment

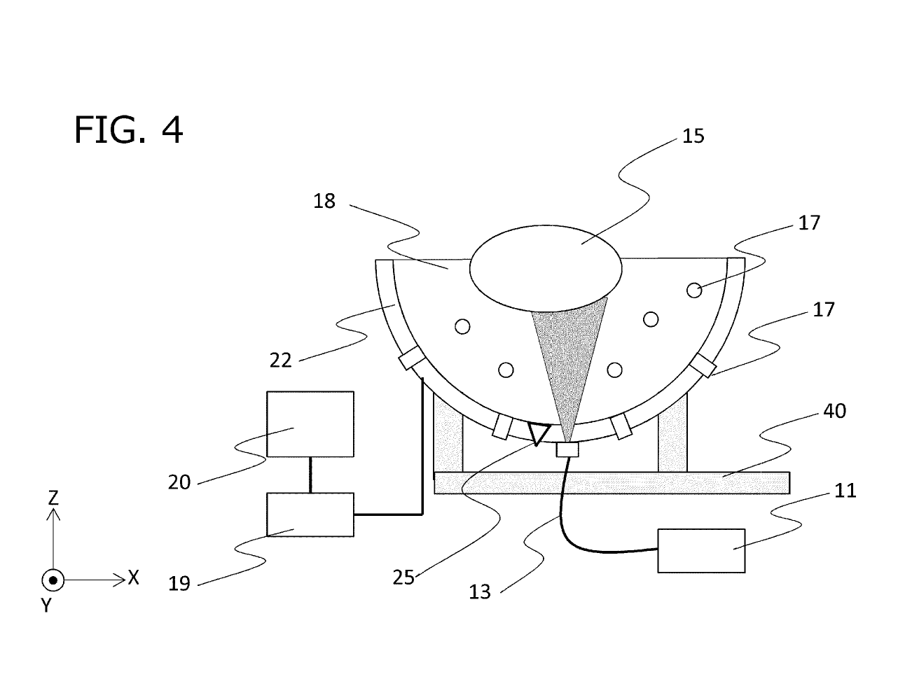

FIG. 4 is a diagram schematically depicting another embodiment of the present invention.

A stage 40 supports the supporter 22. The stage 40 changes the relative position of the supporter 22 with respect to the object 15 during the imaging operation. In this embodiment, the stage 40 is moved so that the supporter 22 moves within the XY plane. The stage 40 and the controller thereof comprise a mover, which relatively moves the support with respect to the object within a movement region.

It is preferable that the stage 40 moves the supporter 22 in a circular motion. The circular motion includes a curvilinear motion similar to an ellipse or circle. Further, it is preferable that the stage 40 moves the supporter 22 such that the coordinates of the supporter 22 in the radial direction, with respect to the center of the movement region, change either in an increasing direction or decreasing direction.

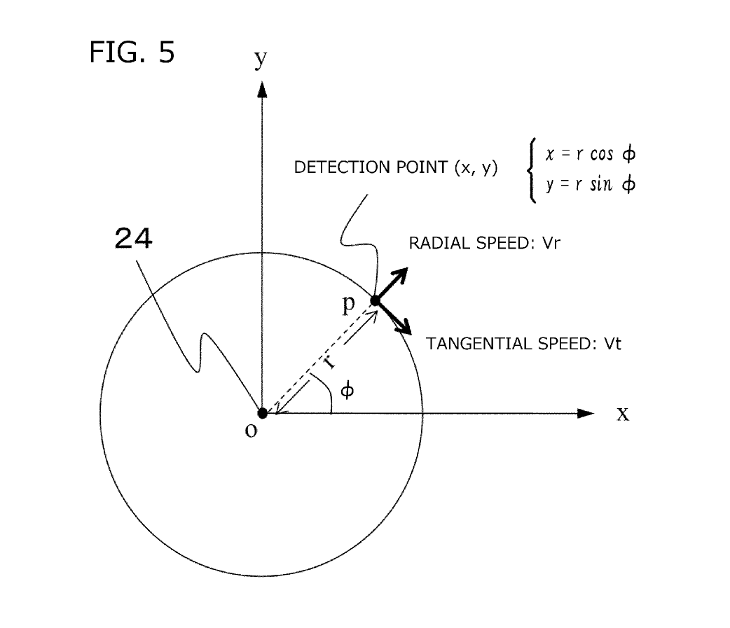

FIG. 5 is a diagram schematically depicting an example of the circular movement. Point o in FIG. 5 is the center 24 of the movement plane, the circle is a movement locus of the position of the supporter 22, and point p is one point on the movement locus of the position of the supporter 22. The position of the supporter 22 according to this embodiment is a point where the perpendicular line drawn from the center of the high resolution region to the movement plane intersects with the supporter 22, and if the supporter 22 is a hemisphere, the polar portion of the hemisphere is the position of the supporter 22. The position of the supporter 22 at the point p has a radial speed: Vr and tangential speed: Vt. The positional coordinates (x, y) at the point p are expressed by the polar coordinate system, as shown in the following Expression (1).

$$\{ \begin{matrix}x = r cos φ \\ y = r sin φ\end{matrix} \qquad [ Math . 5 ]$$

Here r denotes a coordinate (movement radius) in the radial direction, and φ denotes an angle formed by the x axis and a line from the origin to the point p. In this embodiment, the stage 40 moves the supporter 22 such that the coordinate (r) of the position of the supporter 22 in the radial direction on the movement locus changes either in an increasing direction or decreasing direction.

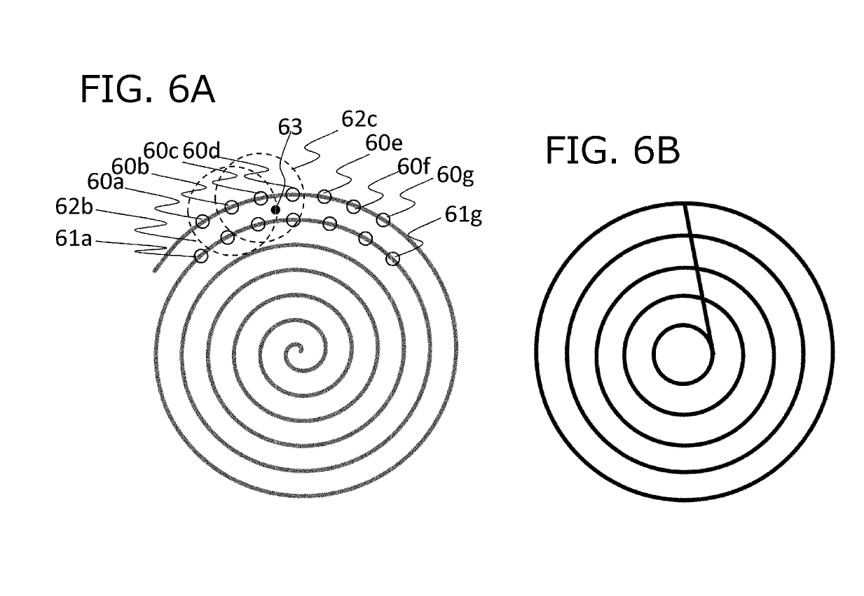

Concrete examples of the movement locus are: a spiral movement locus where the radius changes with time, as shown in FIG. 6A; and a movement locus constituted by a plurality of concentric circles having different radiuses, as shown in FIG. 6B.

The acoustic matching material 18 that fills the container of the supporter 22 receives inertial force by the movement of the supporter 22. If the supporter 22 moves linearly and changes direction repeatedly, the liquid surface may be changed, and ripples may be generated by the inertial force. Because of this, the acoustic matching material 18 may not be filled between the object 15 and the plurality of transducers 17. On the other hand, if the supporter 22 is circularly moved, the acoustic matching material 22 constantly receives a force in the circumferential direction of the circular motion. Therefore in the case of circular motion, compared with the movement pattern generated by the linear motion where direction is changed repeatedly, the change of the liquid surface becomes smooth, and acoustic matching between the object 15 and the plurality of transducers 17 can be easily performed.

As described above, if the supporter 22 is moved circularly, sudden acceleration/deceleration is reduced, and the motion of the acoustic matching material 18 can be controlled. As a result, good acoustic matching can be maintained between the object 15 and the plurality of transducers 17.

It is preferable that the stage 40 moves the supporter 22 such that the tangential speed on the movement path becomes constant. If the light source 11 is a pulsed light source that emits light at a predetermined cycle, the timing of measuring the photoacoustic wave is determined by the repetition frequency of the pulsed light emitted from the light source 11. For example, if the light source 11 having a 10 Hz repetition frequency is used, the photoacoustic wave is generated once every 0.1 seconds. Therefore, if it is assumed that the photoacoustic wave is measured every 0.1 seconds when the tangential speed is constant, the measurement positions are uniformly distributed in the space.

It is also preferable that the stage 40 moves the supporter 22 from outside the movement plane considering acceleration in a direction toward the origin. In other words, if acceleration is high in the initial stage of the movement, the entire apparatus largely shakes, and this shaking may influence the measurement. Therefore the shaking of the apparatus is reduced if the supporter 22 is moved from the outer circumference, where acceleration toward the origin is small, to the inner circumference.

It is also preferable that the stage 40 is a continuous type that moves the supporter 22 continuously, instead of a step and repeat type, which repeatedly moves and stops the supporter 22. Thereby the entire movement time can be decreased, and burdon on the testee can be decreased. Moreover, the influence of the shaking of the apparatus or the shaking of the acoustic matching material 18 can be decreased, since the change in the acceleration of movement is small.

It is preferable that the stage 40 moves the optical transmission system 13 along with the supporter 22 to move the irradiation position of the pulsed light generated from the light source 11. In other words, it is preferable that the stage 40 moves the supporter 22 and the optical transmission system 13 synchronously. Thereby the relationship between the photoacoustic wave measurement position and the light radiation position is constantly maintained, and more homogeneous object information can be acquired. If the object is a human body, the irradiation area of the object is restricted by American National Standards Institute (ANSI) standards. In order to increase the quantity of light that propagates into the object 15, it is preferable to increase the irradiation intensity and the irradiation area, but the irradiation area is limited in terms of cost of the light source or the like. Also even if the light is radiated into a region of which reception sensitivity is low due to the directivity of the transducer, light utilization efficiency is low. In other words, it is not efficient to radiate light to the entire object. If light is always radiated into a high sensitivity region of the plurality of transducers 17, on the other hand, light utilization efficiency is high, hence it is preferable that the stage 40 moves each of the plurality of transducers 17, while maintaining the positional relationship of the plurality of transducers 17 and the optical transmission system 13.

The computer 19 can control the amount of movement, such as the maximum value of the coordinate (r) in the radial direction, the moving speed (radial speed and tangential speed), and the method of changing the coordinate (r) in the radial direction or the like. It is preferable that the maximum value of the coordinate (r) in the radial direction is changed according to the size of the object. For example, if the object is small, the movement of the supporter 22 is controlled so that r becomes relatively small, and as the size of the object is larger, the movement of the supporter 22 is controlled so that r becomes larger, whereby unnecessary measurement time can be reduced.

Furthermore, it is preferable that the photoacoustic apparatus has a size acquisition unit that acquires information on the size and position of the object 15. For example, a CCD or the like, which can acquire information on the shape of the object 15, can be used as the size acquisition unit. The computer 19 may determine the coordinates of the center position in the movement range and the maximum value of the coordinate (r) in the radial direction according to the information on the size and position of the object 15 acquired from the size acquisition unit.

It is also preferable that the photoacoustic apparatus includes an input unit by which the user can specify the movement parameters, such as the maximum value of the coordinate (r) in the radial direction, to the computer 19.

The stage 40 moves the supporter 22 first and then the pulsed light 12 is radiated at a plurality of timings, hence the high resolution region exists at a different position depending on each measurement timing. For example, if the light is radiated at the position 60b, the region 62b becomes the high resolution region, and if the light is radiated at the position 60c, the region 62c becomes the high resolution region. As a result, the high resolution region expands by moving the supporter 22 as described in this embodiment. In this case, it is preferable that the stage 40 moves the supporter 22 such that the plurality of high resolution regions overlaps with each other, in order to reduce dispersion of resolution within the region to be imaged.

When the ultrasound image is acquired, the ultrasound image is reconstructed by repeating transmission and reception of the ultrasound wave synchronizing with the movement of the supporter 22, just like the case of acquiring the photoacoustic image. In this case, the ultrasound image reconstruction described herein below is performed.

For understanding the image reconstruction process, the following definitions are introduced: hi(t): pulse-echo response of each transmit-receive transducer pair. This function can be measured by transmitting an acoustic wave off of a flat, metal plate and recording the resulting echo for the i-th receive transducer.

sij(t): temporal signal recorded at transducer i following each transmit pulse for each bowl position j.
Hi(w): FFT(hi(t)) is the Fourier Transform of the pulse-echo responses, where w is acoustic angular frequency.
Sij(w): FFT(sij(t)) is the Fourier Transform of the recorded temporal signals.

### Fili(w):

$${( \vert \frac{w}{{w}_{c}} \vert )}^{α} \frac{Apodize ( w , {w}_{c} )}{Hi ( w )} \qquad [ Math . 6 ]$$

is a filter function, where w<wC, 1≦α≦2, and we is an upper bandwidth limit to the acoustic angular frequency.

The apodizing function is well known to those skilled in the art of computed tomography and is used to roll off the filter response smoothly at wc. An example function is

$$\frac{1 + cos ( π \frac{w}{{w}_{c}} )}{2} \qquad [ Math . 7 ]$$

s*ij (t)=IFFT[Fili(w)Sij (w)] is the filtered temporal signal recorded at transducer i following each transmit pulse for each bowl position j, where IFFT is the inverse Fourier Transform.
The 3D integrated backscatter image is then computed as:

I(r)=|Σ_{i,j}s*ij(t(r))|*P(r), [Math. 8]

Where “∥” denotes absolute value, P(r) is a 3D smoothing filter, e.g., a 3D Gaussian, and “*” denotes 3D convolution.

By reconstructing the ultrasound image using the ultrasound digital signals acquired while moving the supporter 22 like this, the high resolution region of the ultrasound image is located in a different position depending on each measurement timing, just like the case of the light-induced ultrasound image. As a result, the high resolution region expands. In this case, it is preferable that the stage 40 moves the supporter 22 such that the plurality of high resolution regions overlaps with each other, in order to reduce dispersion of resolution within the region to be imaged.

By using this method for moving the supporter 22 while continuously performing the pulsed light radiation to the object 15 and the ultrasound transmission, the acquisition time for the light-induced ultrasound image and for the ultrasound image can be similar. Then even if the position of the object 15 fluctuates, the relative positions of the light induced ultrasound image and the ultrasound image can be kept close to each other, and high positional alignment can be performed in the case of a superimposed display, for example. Furthermore, the light-induced ultrasound image and the ultrasound image, of which positions are accurately aligned, can be provided over a wide range.

### Another Embodiment

Another embodiment of the present invention will be described with reference to FIG. 6.

The ultrasound wave is transmitted while the supporter 22 is moving at each position 60a, 60b, . . . 60g on the movement path of the supporter 22. Using the signal received at each position, an ultrasound image is reconstructed. For example, if the ultrasound image of the position 63 is reconstructed, the ultrasound digital signals acquired at four positions: 60c, 60d, 61c and 61d are used since the position 63 is in the high resolution region that includes the positions 60c, 60d, 61c and 61d.

The signals acquired at the positions 60c, 60d, 61c and 61d are assumed to be si1, si2, si3 and si4 (i is an element number). Then the ultrasound reconstructed image is calculated as follows.

$$I ( r ) = ( \vert {\sum }_{i , j = 1 , 2} s ⋆ ij ( t ( r ) ) \vert + \vert {\sum }_{i , j = 3 , 4} s ⋆ ij ( t ( r ) ) \vert ) ⋆ P ( r ) \qquad [ Math . 9 ]$$

In other words, the signal levels of the ultrasound digital signals acquired at the positions 60c and 60d are added, and the signal levels of the ultrasound digital signals acquired at the positions 61c and 61d are added, then the absolute values of the respective added results are calculated and these absolute values are added.

In the above processing, signals of which the acquisition time difference is shorter than a predetermined reference value are added, and if the acquisition time difference of signals is longer than the predetermined reference value, the absolute values of the signals are calculated and then these absolute values are added.

If the object 15 is a living body, the position of the object 15 may be changed, or the object 15 may be deformed while acquiring the light-induced ultrasound image and the ultrasound image. In this case, if the signals are simply added, signals may cancel each other out if the signals have a same value with opposite positive and negative signs. This embodiment is for solving this problem, and for signals of which the acquiring time difference is longer than a predetermined reference value, where absolute values thereof are calculated first and these absolute values are added. By this processing, a possibility of signals cancelling each other out can be minimized. And since unintended deletion of signals can be reduced, an image with higher reliability can be provided.

The predetermined reference value may be selected by the operator considering the influence of breathing and pulsation of the living body and other involuntary movements, or may be automatically switched depending on the object of the photoacoustic apparatus. For example, if movement due to breathing and the influence of deformation are considered, the predetermined reference value is preferably set to about 1 second or about 0.5 seconds or less. This is because when the breathing cycle is regarded as 3 seconds, the movement amount is set to be less than the maximum amplitude of deformation caused by breathing. If the influence of pulsation is considered, the predetermined reference value is preferably set to about 300 msec or 150 about msec or less.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of U.S. Provisional Application No. 62/077,369, filed on Nov. 10, 2014, which is hereby incorporated by reference herein in its entirety.

1. An object information acquiring apparatus comprising:
a light source;
an ultrasound transmitting element;
a plurality of transducers each configured to detect a first acoustic wave generated by light, which is from the light source and which is radiated on an object, and output a first electric signal, and detect a second acoustic wave generated by an ultrasound wave, which is transmitted from the ultrasound transmitting element and which is scattered inside the object, and output a second electric signal;
a supporter configured to support the plurality of transducers so that directivity axes of the transducers are concentrated; and
a processor configured to acquire property information on the object based on the first electric signal and the second electric signal respectively.

2. The object information acquiring apparatus according to claim 1, further comprising a mover relatively moving the supporter with respect to the object within a movement region, wherein
the processor acquires the specific information based on the first electric signal and the second electric signal acquired at a plurality of positions within the moving region respectively.

3. The object information acquiring apparatus according to claim 2, wherein
the mover moves the supporter spirally.
